(19) United States
(12) Plant Patent Application Publication
(10) Pub. No.: US 2017/0311502 P1
(43) Pub. Date: Oct. 26, 2017

(54) Broussonetia plant named 'JINFENG'

(50) ;

(71) Applicant: HENAN MINGPIN COLORIZED SEEDLING CO., LTD, Suiping (CN)

(72) Inventor: Huaming Wang, Suiping (CN)

(21) Appl. No.: 15/731,041

(22) Filed: Apr. 11, 2017

(51) Int. Cl. A01H 5/12

(52) U.S. Cl. CPC A01H 5/12
USPC PLT/216

(57) ABSTRACT

‘Jinfeng’ is a distinctive variety of Broussonetia Plant, which is characterized by its color of leaves.

Genus and species: Broussonetia L'Hér. ex Vent.

Variety denomination: Jinfeng.

### BACKGROUND OF THE INVENTION

The present invention relates to a new and distinct variety of Broussonetia plant, which is given the varietal denomination of ‘Jinfeng’.

In April 2008, a mutated Broussonetia plant was found among Broussonetia seedlings in Xinping Village, Suiping County, Henan Province, China. Leaves of the mutated Broussonetia plant kept golden until falling.

In April 2009, the mutated Broussonetia plant was grafted, and characteristics thereof were kept stable.

From 2010-2014, different generations were used for reproducing. No further mutation was observed. The distinct variety was healthy, and characteristics thereof were kept stable.

The new and distinct variety of Broussonetia plant is not commercially available.

### SUMMARY OF THE NEW VARIETY

‘Jinfeng’ is a distinctive variety of Broussonetia plant with deciduous leaves, whose crown is extended. Bark of ‘Jinfeng’ is smooth, light gray or grayish brown, and without cracking. The whole plant contains milk-like sap. Young branches are densely covered with fine hairs. Single-leaves, which grow alternately and sometimes almost oppositely, are thick papery. The leaves are oval-shaped or wide oval-shaped with acute apexes. Bases of the leaves are rounded, subcordate or skewed. The leaves have serrated margins, and have no crack or 3-5 cracks. Upper surfaces of the leaves are rough and covered with hard hairs. Lower surfaces of the leaves are covered with soft hairs. There are 7-8 pairs of lateral veins. In spring, the leaves are golden. In summer, the leaves are yellow-green. In autumn, the leaves turn back to golden and bright. Petioles long 3-5 cm, and are densely covered with thick hairs. Stipules are ovate or oblong, and caduceus. Flowers are male with Catkin inflorescence, flowering from April to May.

### BRIEF DESCRIPTION OF THE DRAWINGS

FIG. 1 is a color photograph of the new invention, ‘Jinfeng’, showing leaves in spring.

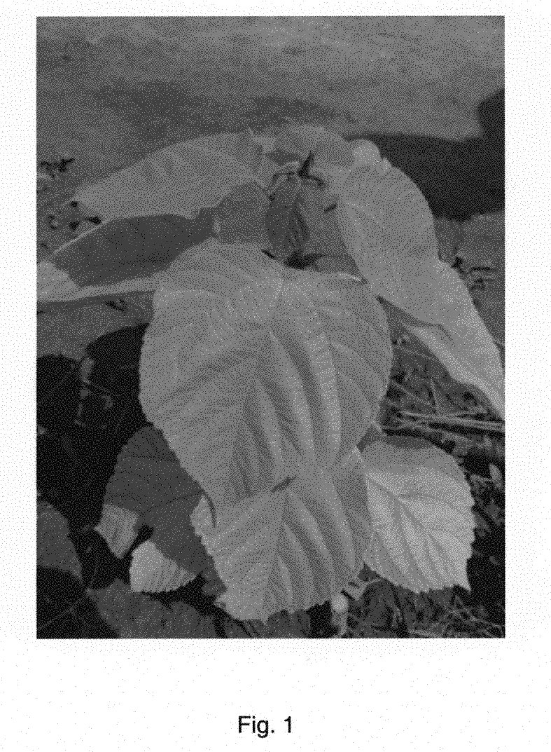

FIG. 2 is a color photograph of the new invention, ‘Jinfeng’, showing leaves in summer.

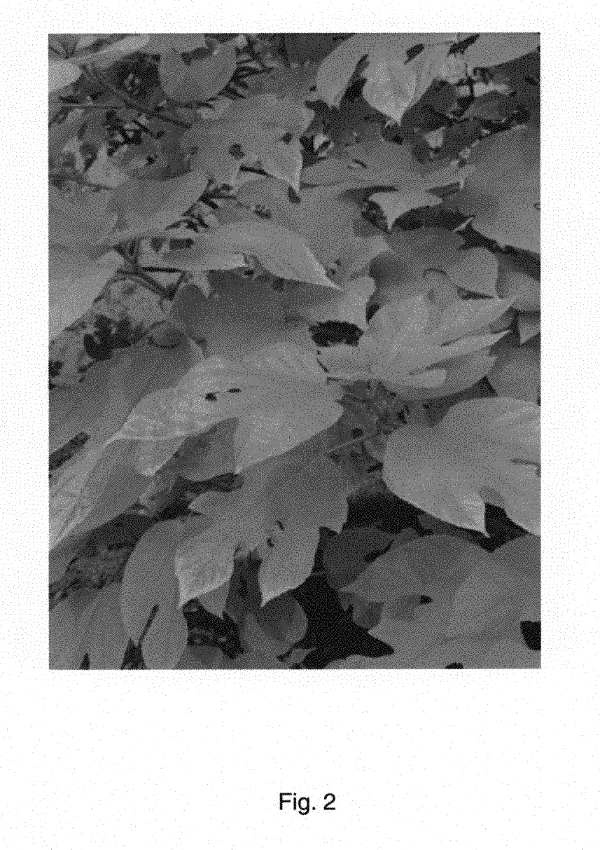

FIG. 3 is a color photograph of the new invention, ‘Jinfeng’, showing leaves in autumn.

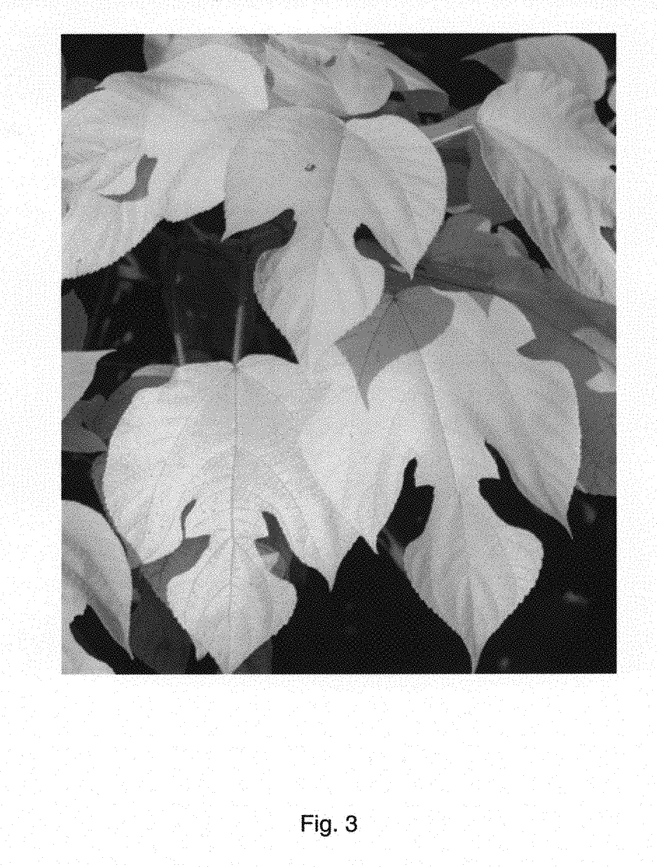

FIG. 4 is a color photograph of the new invention, ‘Jinfeng’, showing upward extending stem.

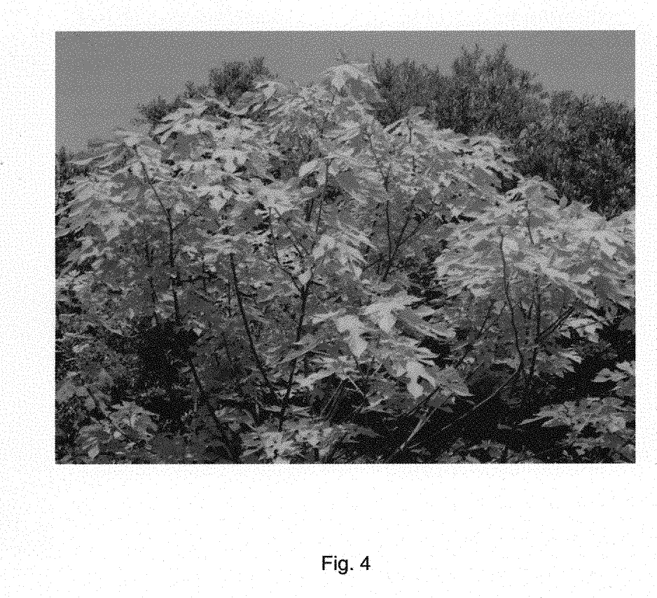

FIG. 5 is a color photograph of the new invention, ‘Jinfeng’, showing the plant in the field.

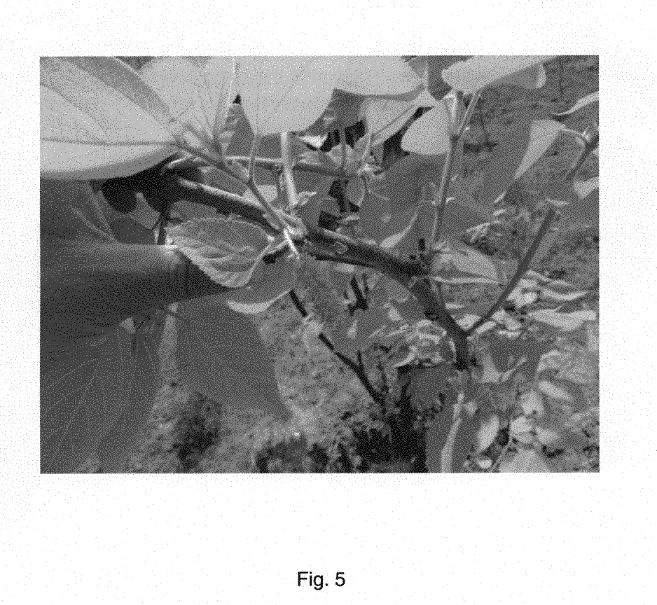

FIG. 6 is a color photograph of the invention, ‘Jinfeng’, showing the appearance of its flowers in spring.

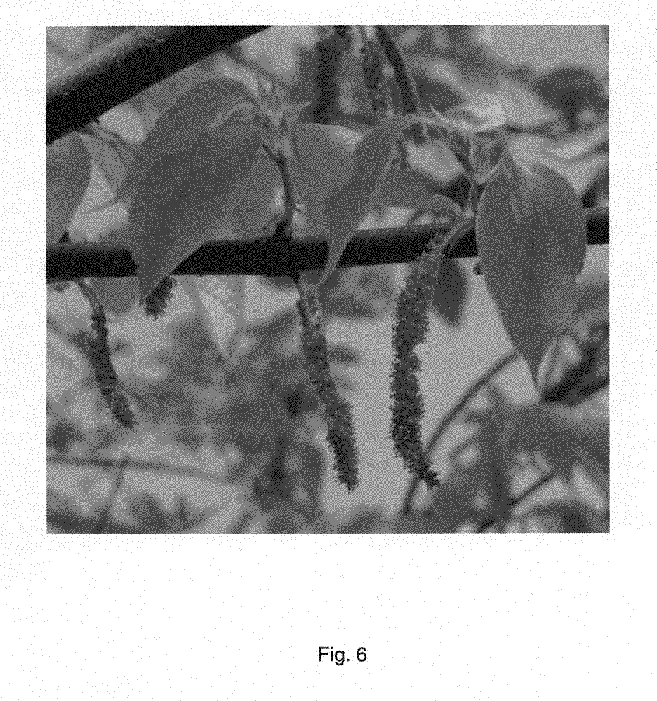

### DETAILED DESCRIPTION

The accompanying colored photographs illustrate the overall appearance of the new and distinct variety of ‘Jinfeng’. The descriptions disclosed herein are based upon observations of the plant growth.

‘Jinfeng’ is a distinctive variety of Broussonetia plant with deciduous leaves, whose crown is extended. Bark of ‘Jinfeng’ is smooth, light gray or grayish brown, and without cracking. The whole plant contains milk-like sap. Young branches are densely covered with fine hairs. Single-leaves, which grow alternately and sometimes almost oppositely, are thick papery. The leaves are oval-shaped or wide oval-shaped with acute apexes. Bases of the leaves are rounded, subcordate or skewed. The leaves have serrated margins, and have no crack or 3-5 cracks. Upper surfaces of the leaves are rough and covered with hard hairs. Lower surfaces of the leaves are covered with soft hairs. There are 7-8 pairs of lateral veins. In spring, the leaves are golden. In summer, the leaves are yellow-green. In autumn, the leaves turn back to golden and bright. Petioles long 3-5 cm, and are densely covered with thick hairs. Stipules are ovate or oblong, and caduceus. Flowers are male with Catkin inflorescence, flowering from April to May.

‘Jinfeng’ is a distinctive variety of Broussonetia plant, which is able to grow in north, center, southwest, and northwest of China, mainly in the Yellow River area, the Yangtze River area and the Pearl River Basin area.

‘Jinfeng’ is a strong phototropism tree species, whose adaptability is particularly strong. The plant has strong resistance to dry-cold and dry and damp-hot climates, and is able to grow in acidic soil, neutral soil and limestone mountain area due to resistance to drought and barren. The plant grows well at both waterside and wetlands. Roots of the plant are shallow and lateral, which are widely distributed and rapidly grow. ‘Jinfeng’ has strong abilities in budding and tillering, with strong resistance to pruning and pollution.

Reproducing methods of ‘Jinfeng’ are mainly graft and cottage. For grafting, Broussonetia plant is used as a rootstock. In spring, cleft or budding with xylem should be adapted for reproducing. In summer, budding is used for reproducing. For cottage, young summer branches are mainly used.

‘Jinfeng’ can be cultivated and rapidly grow in both spring and autumn. During growth, trimming is needed. The plant is not suitable for growing at low areas which are like to suffer water logging.

‘Jinfeng’ rarely suffers from pests and diseases. However, in some particular regions having trunk-attacking pests such as longhorned beetles and jewel beetles, attention should be paid for preventing.

TABLE
comparison between ‘Jinfeng’ and similar species.

| similar species | | features of similar species | features of ‘Jinfeng’ |
|---|---|---|---|
| 1 | paper | dioecism | male |
| | mulberry | green young leaves | yellow |
| | | green mature leaves | yellow-green in summer, |
| | | | golden in autumn |
| 2 | Jinyang | female | male |
| | | smooth upper surface of leaf | rough |
| | | no hair or nearly no hair on | densely cover by hairs |
| | | upper surface of leaf | |

These features and other characteristics of the plant are apparent from the figures.

What is claimed is:

1. A new and distinct variety of Broussonetia Plant named ‘Jinfeng’, substantially as described and illustrated.
